(19) United States
(12) Patent Application Publication
(10) Pub. No.: US 2010/0256046 A1
(43) Pub. Date: Oct. 7, 2010

(54) TREATMENT OF PROLIFERATIVE DISORDERS

(75) Inventors: Stacy SPRINGS, Watertown, MA (US); Mark McKINLAY, Phoenixville, PA (US); Sri CHUNDURU, West Chester, PA (US); Chris BENETATOS, Downingtown, PA (US)

Correspondence Address: BANNER & WITCOFF, LTD., 1100 13th STREET, N.W., SUITE 1200, WASHINGTON, DC, 20005-4051, US

(73) Assignee: TETRALOGIC PHARMACEUTICALS CORPORATION, Malvern, PA (US)

(21) Appl. No.: 12/418,074

(22) Filed: Apr. 3, 2009

(51) Int. Cl. A61K 38/02; A61P 35/00

(52) U.S. Cl. 514/2

(57) ABSTRACT

Inhibitors of cIAP-1 and methods and compositions for treating proliferative disorders.

### CROSS REFERENCE

This Application claims priority from U.S. Provisional Application No. 60/706,649 entitled “PEPTIDOMIMETICS OF SMAC AS cIAP INHIBITORS” filed on Aug. 9, 2005 and U.S. application Ser. No. 11/463,542 entitled “TREATMENT OF PROLIFERATIVE DISORDERS” filed on Aug. 9, 2006.

Apoptosis (programmed cell death) plays a central role in the development and homeostasis of all multi-cellular organisms. Apoptosis can be initiated within a cell from an external factor such as a chemokine (an extrinsic pathway) or via an intracellular event such a DNA damage (an intrinsic pathway). Alterations in apoptotic pathways have been implicated in many types of human pathologies, including developmental disorders, cancer, autoimmune diseases, as well as neurodegenerative disorders. One mode of action of chemotherapeutic drugs is cell death via apoptosis.

Apoptosis is conserved across species and executed primarily by activated caspases, a family of cysteine proteases with aspartate specificity in their substrates. These cysteine containing aspartate specific proteases (“caspases”) are produced in cells as catalytically inactive zymogens and are proteolytically processed to become active proteases during apoptosis. Once activated, effector caspases are responsible for proteolytic cleavage of a broad spectrum of cellular targets that ultimately lead to cell death. In normal surviving cells that have not received an apoptotic stimulus, most caspases remain inactive. If caspases are aberrantly activated, their proteolytic activity can be inhibited by a family of evolutionarily conserved proteins called IAPB (inhibitors of apoptosis proteins).

The IAP family of proteins suppresses apoptosis by preventing the activation of procaspases and inhibiting the enzymatic activity of mature caspases. Several distinct mammalian IAPB including XIAP, c-IAP1, c-IAP2, ML-IAP, NAIP (neuronal apoptosis inhibiting protein), Bruce, and survivin, have been identified, and they all exhibit anti-apoptotic activity in cell culture. IAPB were originally discovered in baculovirus by their functional ability to substitute for P35 protein, an anti-apoptotic gene. IAPB have been described in organisms ranging from Drosophila to human, and are known to be overexpressed in many human cancers. Generally speaking, IAPB comprise one to three Baculovirus IAP repeat (BIR) domains, and most of them also possess a carboxyl-terminal RING finger motif. The BIR domain itself is a zinc binding domain of about 70 residues comprising 4 alpha-helices and 3 beta strands, with cysteine and histidine residues that coordinate the zinc ion. It is the BIR domain that is believed to cause the anti-apoptotic effect by inhibiting the caspases and thus inhibiting apoptosis. XIAP is expressed ubiquitously in most adult and fetal tissues. Overexpression of XIAP in tumor cells has been demonstrated to confer protection against a variety of pro-apoptotic stimuli and promotes resistance to chemotherapy. Consistent with this, a strong correlation between XIAP protein levels and survival has been demonstrated for patients with acute myelogenous leukemia. Down-regulation of XIAP expression by antisense oligonucleotides has been shown to sensitize tumor cells to death induced by a wide range of pro-apoptotic agents, both in vitro and in vivo. Smac/DIABLO-derived peptides have also been demonstrated to sensitize a number of different tumor cell lines to apoptosis induced by a variety of pro-apoptotic drugs.

In normal cells signaled to undergo apoptosis, however, the IAP-mediated inhibitory effect must be removed, a process at least in part performed by a mitochondrial protein named Smac (second mitochondrial activator of caspases). Smac (or, DIABLO), is synthesized as a precursor molecule of 239 amino acids; the N-terminal 55 residues serve as the mitochondria targeting sequence that is removed after import. The mature form of Smac contains 184 amino acids and behaves as an oligomer in solution. Smac and various fragments thereof have been proposed for use as targets for identification of therapeutic agents.

Smac is synthesized in the cytoplasm with an N-terminal mitochondrial targeting sequence that is proteolytically removed during maturation to the mature polypeptide and is then targeted to the inter-membrane space of mitochondria. At the time of apoptosis induction, Smac is released from mitochondria into the cytosol, together with cytochrome c, where it binds to IAPB, and enables caspase activation, therein eliminating the inhibitory effect of IAPB on apoptosis. Whereas cytochrome c induces multimerization of Apaf-1 to activate procaspase-9 and -3, Smac eliminates the inhibitory effect of multiple IAPB. Smac interacts with essentially all IAPB that have been examined to date including XIAP, c-IAP1, c-IAP2, ML-IAP, and survivin. Thus, Smac appears to be a master regulator of apoptosis in mammals.

It has been shown that Smac promotes not only the proteolytic activation of procaspases, but also the enzymatic activity of mature caspase, both of which depend upon its ability to interact physically with IAPB. X-ray crystallography has shown that the first four amino acids (AVPI) of mature Smac bind to a portion of IAPB. This N-terminal sequence is essential for binding IAPB and blocking their anti-apoptotic effects.

Current trends in cancer drug design focus on selective targeting to activate the apoptotic signaling pathways within tumors while sparing normal cells. The tumor specific properties of specific chemotherapeutic agents, such as TRAIL have been reported. The tumor necrosis factor-related apoptosis-inducing ligand (TRAIL) is one of several members of the tumor necrosis factor (TNF) superfamily that induce apoptosis through the engagement of death receptors. TRAIL interacts with an unusually complex receptor system, which in humans comprises two death receptors and three decoy receptors. TRAIL has been used as an anti-cancer agent alone and in combination with other agents including ionizing radiation. TRAIL can initiate apoptosis in cells that overexpress the survival factors Bc1-2 and Bc1-XL, and may represent a treatment strategy for tumors that have acquired resistance to chemotherapeutic drugs. TRAIL binds its cognate receptors and activates the caspase cascade utilizing adapter molecules such as TRADD. TRAIL signaling can be inhibited by overexpression of cIAP-1 or 2, indicating an important role for these proteins in the signaling pathway. Currently, five TRAIL receptors have been identified. Two receptors TRAIL-R1 (DR4) and TRAIL-R2 (DR5) mediate apoptotic signaling, and three non-functional receptors, DcR1, DcR2, and osteoprotegerin (OPG) may act as decoy receptors. Agents that increase expression of DR4 and DR5 may exhibit synergistic anti-tumor activity when combined with TRAIL.

The basic biology of how IAP antagonists work suggests that they may complement or synergize with other chemotherapeutic/anti-neoplastic agents and/or radiation. Chemotherapeutic/anti-neoplastic agents and radiation would be expected to induce apoptosis as a result of DNA damage and/or the disruption of cellular metabolism.

Inhibition of the ability of a cancer cell to replicate and/or repair DNA damage will enhance nuclear DNA fragmentation and thus will promote the cell to enter the apoptotic pathway. Topoisomerases, a class of enzymes that reduce supercoiling in DNA by breaking and rejoining one or both strands of the DNA molecule, are vital to cellular processes, such as DNA replication and repair. Inhibition of this class of enzymes impairs the cells ability to replicate as well as to repair damaged DNA and activates the intrinsic apoptotic pathway.

The main pathways leading from topoisomerase-mediated DNA damage to cell death involve activation of caspases in the cytoplasm by proapoptotic molecules released from mitochondria, such as Smac. The engagement of these apoptotic effector pathways is tightly controlled by upstream regulatory pathways that respond to DNA lesions-induced by topoisomerase inhibitors in cells undergoing apoptosis. Initiation of cellular responses to DNA lesions-induced by topoisomerase inhibitors is ensured by the protein kinases which bind to DNA breaks. These kinases (non-limiting examples of which include Akt, JNK and P38) commonly called “DNA sensors” mediate DNA repair, cell cycle arrest and/or apoptosis by phosphorylating a large number of substrates, including several downstream kinases.

Platinum chemotherapy drugs belong to a general group of DNA modifying agents. DNA modifying agents may be any highly reactive chemical compound that bonds with various nucleophilic groups in nucleic acids and proteins and cause mutagenic, carcinogenic, or cytotoxic effects. DNA modifying agents work by different mechanisms, disruption of DNA function and cell death; DNA damage/the formation of cross-bridges or bonds between atoms in the DNA; and induction of mispairing of the nucleotides leading to mutations, to achieve the same end result: Three non-limiting examples of a platinum containing DNA modifying agents are cisplatin, carboplatin and oxaliplatin.

Cisplatin is believed to kill cancer cells by binding to DNA and interfering with its repair mechanism, eventually leading to cell death. Carboplatin and oxaliplatin are cisplatin derivatives that share the same mechanism of action. Highly reactive platinum complexes are formed intracellularly and inhibit DNA synthesis by covalently binding DNA molecules to form intrastrand and interstrand DNA crosslinks.

Non-steroidal anti-inflammatory drugs (NSAIDs) have been shown to induce apoptosis in colorectal cells. NSAIDS appear to induce apoptosis via the release of Smac from the mitochondria (PNAS, Nov. 30, 2004, vol. 101:16897-16902). Therefore, the use of NSAIDs in combination with certain IAP Antagonists would be expected to increase the activity each drug over the activity of either drug independently.

The process of drug discovery typically entails screening of compounds to identify those compounds that have a desirable biological activity, e.g., binding to a certain receptor or other protein, and then, on the basis of such activity, identifying the compound as a lead for further development. Such further development can be, e.g., by chemical modification of the compound to improve its properties (sometimes referred to as lead optimization) or by putting the compound through other tests and analyses to profile the compound and thereby to further assess its potential as a drug development candidate.

At some point, if the process is successful, a compound is then selected for human clinical trials, which are designed, ultimately, to demonstrate safety and efficacy to a level of acceptability to a drug regulatory agency. A drug regulatory agency is a governmental, or quasi-governmental, agency empowered to receive and review applications for approval to market a drug. Examples include the U.S. Food and Drug Administration in the U.S. (“FDA”), the European Agency for the Evaluation of Medicines in the European Union (“EMEA”), and the Ministry of Health in Japan (“MOH”).

The applicant for approval to market a drug submits information and data relating to the safety and efficacy of the compound for which approval is sought. Such data can include data indicating the mechanism by which the compound causes a particular pharmacological result. So, for example, the applicant may submit data showing that the compound binds to a given ligand.

### SUMMARY OF THE INVENTION

The present invention provides methods of discovering compounds for development as agents useful in the treatment of proliferative disorders and to related methods of obtaining regulatory approval therefor and to treating patients therewith, as well as to pharmaceutical compositions useful in such methods.

### BRIEF DESCRIPTION OF THE DRAWING

FIG. 1 are dose response curves showing TRAIL sensitivity/resistance in SK-OV-3^{S/R} cells

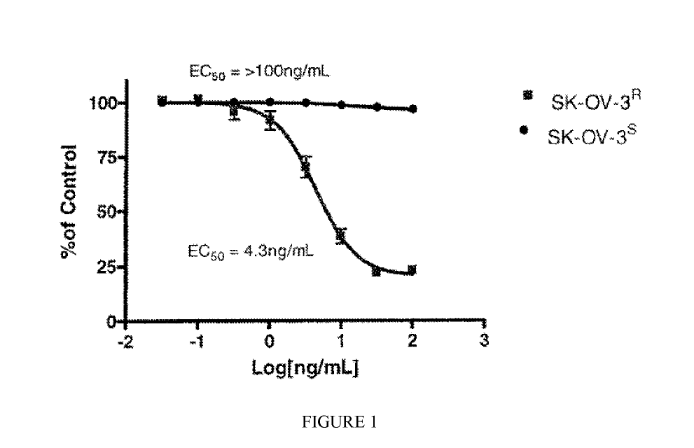

### DESCRIPTION OF ILLUSTRATIVE EMBODIMENTS OF THE INVENTION

This invention relates to the discovery that compounds that bind and thereby degrade cIAP-1 hereinafter referred to as cIAP-1 Antagonists, are particularly useful for the treatment of proliferative disorders. In one aspect of the invention, such compounds are useful in the treatment of cancers, such as, but not limited to, bladder cancer, breast cancer, prostate cancer, lung cancer, pancreatic cancer, gastric cancer, colon cancer, ovarian cancer, renal cancer, hepatoma, melanoma, lymphoma, sarcoma, and combinations thereof. In another aspect, such compounds act as chemopotentiating agents. The term “chemopotentiating agent” refers to an agent that acts to increase the sensitivity of an organism, tissue, or cell to a chemical compound or treatment, namely, “chemotherapeutic agents” or “chemo drugs” or radiation treatment.

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erythematosus or rheumatoid arthritis.

The pathogenic cells can be those of any proliferative autoimmune disease or diseases, which cells are resistant to apoptosis due to the expression of cIAPs. Examples of such autoimmune diseases are collagen diseases such as rheumatoid arthritis, systemic lupus erythematosus, Sharp's syndrome, CREST syndrome (calcinosis, Raynaud's syndrome, esophageal dysmotility, telangiectasia), dermatomyositis, vasculitis (Morbus Wegener's) and Sjogren's syndrome, renal diseases such as Goodpasture's syndrome, rapidly-progressing glomerulonephritis and membrano-proliferative glomerulonephritis type II, endocrine diseases such as type-I diabetes, autoimmune polyendocrinopathy-candidiasis-ectodermal dystrophy (APECED), autoimmune parathyroidism, pernicious anemia, gonad insufficiency, idiopathic Morbus Addison's, hyperthyreosis, Hashimoto's thyroiditis and primary myxedema, skin diseases such as pemphigus vulgaris, bullous pemphigoid, herpes gestationis, epidermolysis bullosa and erythema multiforme major, liver diseases such as primary biliary cirrhosis, autoimmune cholangitis, autoimmune hepatitis type-1, autoimmune hepatitis type-2, primary sclerosing cholangitis, neuronal diseases such as multiple sclerosis, myasthenia gravis, myasthenic Lambert-Eaton syndrome, acquired neuromyotony, Guillain-Barré syndrome (Muller-Fischer syndrome), stiff-man syndrome, cerebellar degeneration, ataxia, opsoklonus, sensoric neuropathy and achalasia, blood diseases such as autoimmune hemolytic anemia, idiopathic thrombocytopenic purpura (Morbus Werlhof), infectious diseases with associated autoimmune reactions such as AIDS, Malaria and Chagas disease.

In certain proliferative disorders, e.g., in certain types of cancer, the aberrant regulation of apoptosis associated with the disorders can be due to a greater extent by cIAP-1 activity than by XIAP activity, notwithstanding that inhibition of apoptosis by XIAP may also be a factor in the disorder. In this case, such patients are preferentially selected for treatment with compounds that preferentially bind and degrade cIAP-1 relative to XIAP, because treatment with such compound will be more effective than treatment with a compound that preferentially binds XIAP.

Compositions useful in the practice of the invention encompass pharmaceutical compositions comprising an effective amount (i.e., an amount that when administered over a full course of therapy is effective in inhibiting disease progression and/or causing regression of disease symptoms) of a cIAP-1 Antagonist, i.e., an IAP antagonist, that binds cIAP-1, in a dosage form and a pharmaceutically acceptable carrier. Another embodiment of the present invention are compositions comprising an effective amount of such cIAP-1 Antagonist in a dosage form and a pharmaceutically acceptable carrier, in combination with a chemotherapeutic and/or radiotherapy, wherein the cIAP-1 Antagonist inhibits the activity of an Inhibitor of Apoptosis protein (IAP), thus promoting apoptosis and enhancing the effectiveness of the chemotherapeutic and/or radiotherapy.

Smac mimetics, i.e., small molecules that mimic the binding activity of the four N-terminal amino acids of mature Smac, are disclosed, e.g., in WO04005248, WO04007529, WO05069894, WO05069888, WO05097791, WO06010118, WO06069063, US20050261203, US20050234042, US20060014700, US2006017295, US20060025347, US20050197403, and U.S. application Ser. No. 11/363,387 filed Feb. 27, 2006, all of which are incorporated herein by reference as though fully set forth.

Compounds of the structures disclosed therein can be screened for cIAP-1 binding affinity or degradation, or both, and selected or rejected for further development on the basis thereof Preferably, such compounds have greater affinity for cIAP-1 than for other IAPB, e.g., they have greater affinity for cIAP-1 than for XIAP. Preferably, the difference in relative affinities as measured by binding constants is at least 3-fold higher for cIAP-1 than for XIAP. More preferably, the binding affinity is at least about an order of magnitude greater, i.e., at least about 10-fold greater, and more preferably is at least about two orders of magnitude greater, i.e., at least about 100-fold greater.

“Mimetics” or “peptidomimetics” are synthetic compounds having a three-dimensional structure (i.e. a “core peptide motif”) based upon the three-dimensional structure of a selected peptide.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24, 243-252, 1989). Certain peptidomimetic compounds are based upon the amino acid sequence of the peptides of the invention. Often, peptidomimetic compounds are synthetic compounds having a three-dimensional structure (i.e. a “peptide motif”) based upon the three-dimensional structure of a selected peptide. The peptide motif provides the peptidomimetic compound with the desired biological activity, i.e., binding to IAP, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life.

Mimetic, specifically, peptidomimetic design strategies are readily available in the art and can be easily adapted for use in the present invention (see, e.g., Ripka & Rich, Curr. Op. Chem. Biol. 2, 441-452, 1998; Hruby et al., Curr. Op. Chem. Biol. 1, 114-119, 1997; Hruby & Balse, Curr. Med. Chem. 9, 945-970, 2000). One class of mimetic mimics a backbone that is partially or completely non-peptide, but mimics the peptide backbone atom-for-atom and comprises side groups that likewise mimic the functionality of the side groups of the native amino acid residues. Several types of chemical bonds, e.g. ester, thioester, thioamide, retroamide, reduced carbonyl, dimethylene and ketomethylene bonds, are known in the art to be generally useful substitutes for peptide bonds in the construction of protease-resistant peptidomimetics. Another class of peptidomimetics comprises a small non-peptide molecule that binds to another peptide or protein, but which is not necessarily a structural mimetic of the native peptide. Yet another class of peptidomimetics has arisen from combinatorial chemistry and the generation of massive chemical libraries. These generally comprise novel templates which, though structurally unrelated to the native peptide, possess necessary functional groups positioned on a nonpeptide scaffold to serve as “topographical” mimetics of the original peptide (Ripka & Rich, 1998, supra).

For example, the IAP-binding peptides of the invention may be modified to produce peptide mimetics by replacement of one or more naturally occurring side chains of the 20 genetically encoded amino acids, or D amino acids with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties.

The present invention provides compounds which bind to cIAP-1. Stereoisomers of the mimetic compounds described herein are also encompassed in the present invention. The invention also provides methods of using these mimetics to modulate apoptosis and further for therapeutic purposes.

Binding Affinities and MTT

To illustrate this invention, Compounds A through R were synthesized and tested in a biochemical binding assay using purified BIR-3 domains of XIAP and c-IAP-1.

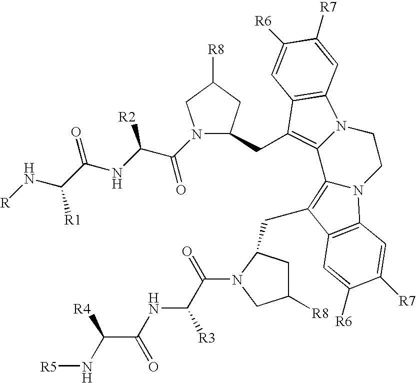

TABLE 1

| GT | | | | | | | | | | | | |
|---|---|---|---|---|---|---|---|---|---|---|---|---|
| Number | Entry | R | R1 | R2 | X | W | R3 | R4 | R5 | R6 | R7 | R8 |
| 13011 | A | Me | Me | sBu | Na | Na | sBu | Me | Me | F | H | H |

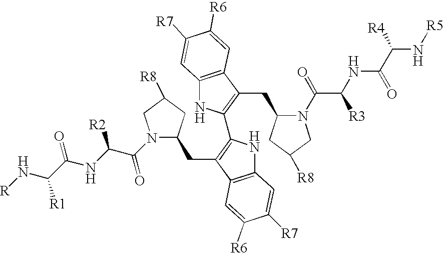

TABLE 2

| GT | | | | | | | | | | | | |
|---|---|---|---|---|---|---|---|---|---|---|---|---|
| Number | Entry | R | R1 | R2 | X | W | R3 | R4 | R5 | R6 | R7 | R8 |
| 13072 | B | Me | Me | 2R- | Na | Na | 2R- | Me | Me | H | F | S—OH |
| | | | | EtO(Me) | | | EtO(Me) | | | | | |
| 13178 | H | Me | Me | 2R- | Na | Na | 2R- | Me | Me | Me | H | S—OH |
| | | | | Et(OMe) | | | Et(OMe) | | | | | |

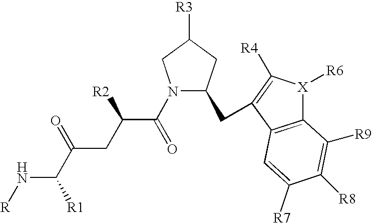

TABLE 3

| GT | | | | | | | | | | | |
|---|---|---|---|---|---|---|---|---|---|---|---|
| Number | Entry | R | R1 | R2 | X | R3 | R4 | R6 | R7 | R8 | R9 |
| 12698 | I | H | H | iPr | O | S-PhO | H | Na | H | H | H |
| 12917 | P | Me | Me | tBu | N | H | 4—CO₂Me- | (CH₂CH₂O)₃Me | H | F | H |
| | | | | | | | phenyl | | | | |
| 12919 | Q | Me | Me | tBu | N | H | 4—F-phenyl | (CH₂CH₂O)₃Me | H | F | H |
| 12920 | R | Me | Me | tBu | N | H | 4-(1- | (CH₂CH₂O)₃Me | H | F | H |
| | | | | | | | morpholino)- | | | | |
| | | | | | | | phenyl | | | | |
| 13103 | F | Me | Me | iPr | N | S—OAc | H | H | H | H | H |
| 13102 | E | Me | Me | tBu | N | S—OAc | H | H | H | H | H |
| 13101 | D | Me | Me | 1R- | N | S—OAc | H | H | H | H | H |
| | | | | EtOH | | | | | | | |
| 13107 | C | Me | Me | 1R- | N | H | H | H | Me | H | H |
| | | | | EtOH | | | | | | | |
| 13105 | G | Me | Me | iPr | N | H | H | H | Me | H | H |

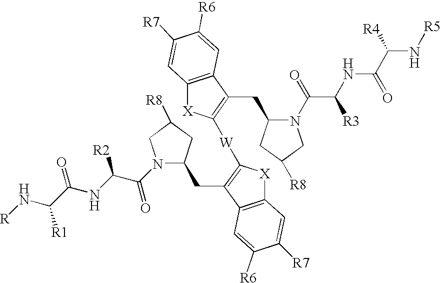

TABLE 4

| GT | | | | | | | | | | | | |
|---|---|---|---|---|---|---|---|---|---|---|---|---|
| Number | Entry | R | R1 | R2 | X | W | R3 | R4 | R5 | R6 | R7 | R8 |
| 12726 | J | H | Me | iPr | O | 1,4- | iPr | Me | H | H | H | H |
| | | | | | | phenyl | | | | | | |
| 12983 | M | Me | Me | tBu | NH | 1,4- | tBu | Me | Me | H | H | (S)—OH |
| | | | | | | phenyl | | | | | | |

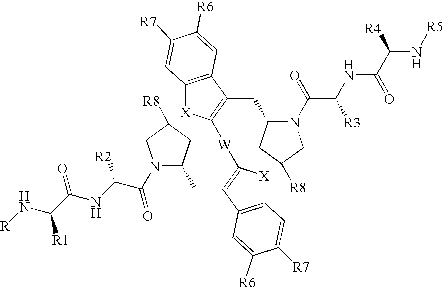

TABLE 5

| GT | | | | | | | | | | | | |
|---|---|---|---|---|---|---|---|---|---|---|---|---|
| Number | Entry | R | R1 | R2 | X | W | R3 | R4 | R5 | R6 | R7 | R8 |
| 12877 | L | H | Me | iPr | O | 1,4- | iPr | Me | H | H | H | H |
| | | | | | | phenyl | | | | | | |

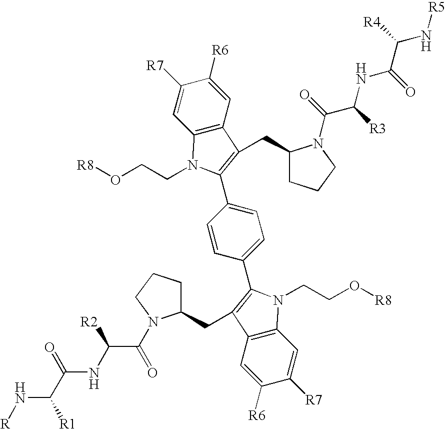

TABLE 6

| GT | | | | | | | | | | | | |
|---|---|---|---|---|---|---|---|---|---|---|---|---|
| Number | Entry | R | R1 | R2 | X | W | R3 | R4 | R5 | R6 | R7 | R8 |
| 12924 | O | Me | Me | iPr | na | na | iPr | Me | Me | H | F | Ac |
| 12911 | N | Me | Me | tBu | na | na | tBu | Me | Me | H | F | H |

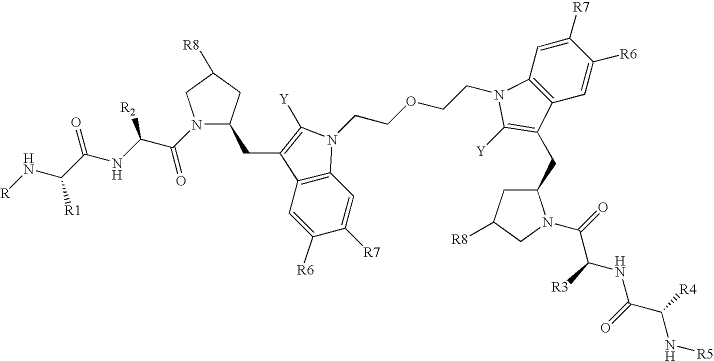

TABLE 7

| GT | | | | | | | | | | | |
|---|---|---|---|---|---|---|---|---|---|---|---|
| Number | Entry | R | R1 | R2 | Y | R3 | R4 | R5 | R6 | R7 | R8 |
| 12794 | K | Me | Me | cHex | H | cHex | Me | Me | H | H | H |

Table 8. IAP antagonists bind (IC₅₀) to BIR-3 domain of cIAP-1 with a higher Binding constants were measured using fluorescence polarization as described before (Zaneta Nikolovska-Coleska et. al., (2004) Analytical Biochemistry, 332, 261-273). Briefly, test peptides at various concentrations for binding measurements were mixed with 5 nM fluorescently labeled peptide (AbuRPF-K(5-Fam)-NH2; FP peptide) and 40 nM of XIAP-Bir3, and cIAP1-BIR3 for 15 min at room temperature (approximately 22° C.) in 100 μL of 0.1M Potassium Phosphate buffer, pH 7.5 containing 100 μg/ml bovine γ-globulin. Following incubation, the polarization values (mP) were measured on a Victor²V using a 485 nm excitation filter and a 535 nm emission filter. IC₅₀ values (Table I) were determined from the plot using nonlinear least-squares analysis using GraphPad Prism.

We also tested the ability of these compounds to inhibit the growth of an ovarian cancer cell line, SK-OV-3 (Table 1). The MTT assay is an example of an assay that has been used for measuring cell growth as previously described (Hansen, M. B., Nielsen, S. E., and Berg, K. (1989) J. Immunol. Methods 119, 203-210) and incorporated herein by reference in its entirety. Briefly, SK-OV-3 cells were seeded in 96-well plates in McCoy's medium containing 10% fetal bovine serum albumin (10,000 per well) and incubated overnight at 37° C. Next day, test compounds were added at various concentrations (0.003-10 μM) and the plates were incubated at 37° C. for an additional 72 hrs. This incubation time was optimal for measuring inhibitory effects of different analogs. 50 microliters of 5 mg/mL MTT reagent to each well was added and the plates were incubated at 37° C. for 3 hours. At the end of incubation period, 50 microliters of DMSO was added to each well to dissolve cells and the optical density (OD) of the wells was measured with a microplate reader (Victor² 1420, Wallac, Finland) at 535 nm. Cell survival (CS) was calculated by the following equation:

CS=(OD treated well/mean OD control wells)×100%.

The EC₅₀ (Table 1), defined as the drug concentration that results in 50% CS, was derived by calculating the point where the dose-response curve crosses the 50% CS point using GraphPad Prism.

affinity than to XIAP.

| | XIAP IC₅₀ | cIAP-1 IC₅₀ | MTT EC₅₀ |
|---|---|---|---|
| Compound | (μM) | (μM) | (μM) |
| AVPI | ++ | +++ | ND |
| AVPF | +++ | +++ | ND |
| A | +++ | ++++ | ++++ |
| B | +++ | ++++ | ++++ |
| C | ++ | ++++ | ++++ |
| D | − | − | ND |
| E | ++ | ++++ | +++ |
| F | +++ | ++++ | +++ |
| G | +++ | ++++ | +++ |
| H | ++ | +++ | ++++ |
| I | ++ | ++++ | ++++ |
| J | ++ | ++ | ++ |
| K | ++ | +++ | +++ |
| L | ++ | +++ | ++++ |
| M | +++ | +++ | +++ |
| N | +++ | +++ | ++++ |
| O | − | − | − |
| P | +++ | +++ | ++++ |
| Q | +++ | +++ | ++++ |
| R | +++ | +++ | +++ |

++++ = <0.01 μM;
+++ = ≧0.01-0.1 μM;
++ = >0.1 μM;
− = >1 μM;
ND = not determined

The homology among the XIAP and cIAP-1 BIR3 domains is high. It is not surprising, therefore, that IAP antagonists that are specifically synthesized to bind to XIAP also bind to cIAP-1. However, the binding data show that certain IAP antagonists bind to cIAP-1 three to over 100-fold more tightly than to XIAP.

IAP Degradation

SKOV3 cells were passed into six 60×15 mm tissue culture dishes 2 days before experiment. Cells appeared to be ˜80% confluent at time of harvest. A freshly prepared solution of 100 nM compound (B or Q) in 10% FBS/90% McCoys 5a (medium A) was used for each time point. This solution was prepared by diluting 1 μl of a 10 mM stock solution of compound (B or Q) DMSO into 10 mL of medium A to generate a 1 μM solution. A 10-fold dilution of this solution into medium A gave the 100 nM working solution. Cells were treated at 0.5, 2, 4, 6 and 8 hours before lysis for western blot analysis by removal of existing medium and addition of 3 mL of the freshly prepared 100 nM solution of compound (B or Q) in medium A.

Western blot analysis was carried out using standard technique. Briefly, cells were lysed using the MPER mammalian cell lysis solution (Bio-Rad #78503) to which 10 μl/mL of a 100× solution of HALT protease inhibitor cocktail (Bio-Rad #78410) has been added. To each dish of cells, 200 μl of the lysis solution plus protease inhibitors is added. The cells in each dish are scraped using a cell scraper and allowed to incubate with the reagent for 10 minutes. The lysates were transferred to pre-chilled microfuge tubes and spun for 20 minutes at 15,000×g at 4° C. The supernatant was transferred to a clean, chilled microfuge tube.

Next, the total protein content of the lysates was determined using the BCA Protein Assay according to the manufacturer's protocol and using interpolation from a standard curve generated with BSA.

The samples were normalized for protein content during preparation for gel electrophoresis. The samples were prepared using 2× Laemmli Sample buffer to which 200 mM DTT was added. The samples were loaded onto 4-15%-HCl polyacrylamide gels (10 lanes, 50 μl wells) and electrophoresis performed at 200 V for 35 minutes in 25 mM Tris, 192 mM Glycine and 0.1% w/v SDS pH 8.3. For each protein probed a separate gel/blot was used for it and it's loading control only. No stripping and reprobing for IAPB was done.

Gels were removed from cartridge and incubated in transfer buffer for at least 15 minutes. Transfer buffer was prepared by mixing 100 mL of 10× Transfer buffer (24.2 g Tris base, 112.6 g glycine in 1L water), 200 mL of methanol and 700 mL of water.

A piece of PVDF was cut to the size of the gel and briefly pre-wet in methanol before soaking in transfer buffer. Filter paper was also cut to the exact size of the membrane and gel and wet in transfer buffer. Fiber pads were also wet. A sandwich consisting of fiber pad, filter paper, gel, membrane, filter paper, fiber pad was assembled. After placing the last piece of filter paper, a glass tube was rolled over the sandwich to remove any air bubbles. The bracket containing the sandwich was closed, locked and placed into the transfer unit with the membrane side facing the positive side of the chamber. A stir bar and Bio-Ice unit were placed in the chamber.

The unit was filled with transfer buffer that had been pre-chilled to 4° C. and a stir bar was added. Buffer stirred while transferring at 100V, 200 mA (max) for 75 minutes.

The back sides of the blots were annotated with pen or pencil and the blots were blocked in 5% w/v non-fat dry milk in TBS-T for 3 hrs at room temperature. The blots were placed in primary antibody solution overnight at 4° C. degC (anti-XIAP R&D Systems Cat #MAB822, lot DYJ01; anti-cIAP-1 R&D Systems Cat #AF8181, lot KHSO1). The blots were washed with at least 5×100 mL of TBS-T and then were incubated for 1 hr at room temperature with the appropriate secondary antibody (anti-mouse-HRP for XIAP blot and anti-goat-HRP for cIAP-1 and cIAP-2; ImmunoPure Goat Anti-Mouse IgG(H+L)-Peroxidase conjugated Pierce Biotechnology (Cat #31430) Lot GI964019; Anti-goat IgG-HRP antibody R&D Systems Cat #HAF109, lot FKA09).

The blots were washed with 5×100 mL of TBS-T, changing containers frequently. For detection, the Amersham ECL kit and ECL Hyperfilm were used according to the manufacturer's specifications.

The time course analysis of cIAP-1 and XIAP disappearance showed that cIAP-1 was completely degraded within the first hour of IAP antagonist treatment whereas XIAP does not begin to degrade until 6 to 8 hours. Thus, preferred cIAP-1 Antagonists of the invention will, following administration to a patient, cause cIAP degradation to occur more rapidly than XIAP degradation, e.g., at a rate that is 2-fold, 3-fold, 4-fold, 5-fold, 6-fold, 7-fold, 8-fold, 9-fold, 10-fold, or more, faster than the rate of degradation of XIAP.

Effect of Proteasome Inhibitor

SK-OV-3 cells in McCoy's medium containing 10% fetal bovine serum albumin were treated with cIAP-1 Antagonists (Compounds B and Q) for 20 hrs in the presence and absence of bortezomib, a proteasome inhibitor.

Cells were harvested after trypsinization by centrifuging at 2000 rpm for 10 min. The cell pellet was washed with PBS and lysed with RIPA to disrupt the cell membrane. The lysate after centrifugation was loaded onto a 5-20% polyacrylamide gel to separate the proteins. Western blot was carried out using standard techniques and probed for XIAP and cIAP-1 proteins as described above. Cells treated with Compounds B and Q in the absence of bortezomib, a proteasome inhibitor showed complete disappearance of both cIAP-1 and XIAP.

The degradation of cIAP-1 can be abrogated with bortezomib. This indicates that the degradation is mediated by ubiquitination possibly due to crosslinking of the RING domains of XIAP and cIAP-1.

TRAIL Synergy

Two distinct cIAP-1 Antagonists were chosen for this experiment in which Compound I binds to cIAP-1 117-fold more tightly than to XIAP while compound S binds to XIAP and cIAP-1 with comparable affinity (Table 2). MTT assays were setup by testing a matrix of concentrations of both drugs.

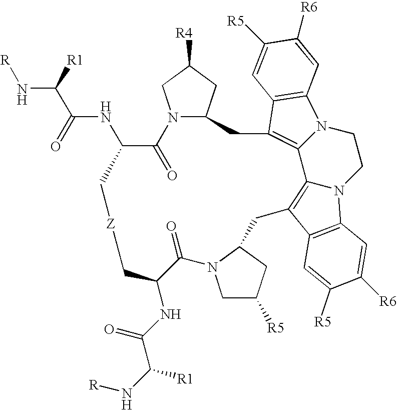

TABLE 9

| Entry | R | R1 | R2 | X | Z | R3 | R4 | R5 | R6 | R7 | R8 |
|---|---|---|---|---|---|---|---|---|---|---|---|
| S | Me | Me | Na | Na | —CH₂CH₂— | Na | OH | OH | F | Na | Na |

These two compounds were tested for synergistic toxicity in MDA-MB231 cells with TRAIL. We observed that the amount of synergistic toxicity as measured by synergy volume using MACSYNERGY II program was identical.

Compounds S and I were also tested for synergistic toxicity in OVCAR-3 cell line with a topoisomerase I inhibitor, SN-38, an active moiety of irinotecan was used. The synergistic volume again was comparable suggesting that cIAP-1 is playing a more significant role than XIAP in showing synergistic toxicity.

TABLE 10
IAP antagonists that bind more tightly to the BIR-3 domains of
cIAP-1 than to XIAP show equivalent cell killing of SKOV-3 cells
and equivalent synergistic toxicity with TRAIL and SN-38

| | IC₅₀ | IC₅₀ | MTT |
|---|---|---|---|
| Compound | (XIAP) | (cIAP-1) | (SKOV-3) |
| S | ++++ | ++++ | ++++ |
| I | ++ | ++++ | ++++ |

Another unexpected observation we made was with respect to TRAIL sensitivity. IAP Antagonist-resistant SK-OV-3 cells (SK-OV-3^{R}) were generated by exposing the parental SK-OV-3 cells (SK-OV-3^{S}) to an IAP antagonist compound at a concentration that kills 95% of cells. Three days later, viable cells were transferred to a fresh flask and grown to confluency. Two weeks later, the cells were tested for IAP Antagonist sensitivity in an MTT assay as described above and as expected, found these cells to be resistant to IAP Antagonist cytotoxicity.

SK-OV-3^{R} cells were subsequently tested for TRAIL sensitivity in an MTT assay and were found to be sensitive to TRAIL while the SK-OV-3^{S} cells are resistant to TRAIL (data below).

Similar results were also observed in a breast cancer cell line: MDA-MB-231.

Western blot analysis of cell lysates obtained from both SK-OV-3^{S} and SK-OV-3^{R} cell lines were carried out as described above. Cell lysate from SK-OV-3^{S} cell line showed the presence of cIAP-1 protein while no cIAP-1 band was observed in the cell lysate obtained from SK-OV-3^{R} cell line. These results suggest that cIAP-1 is playing an important role in TRAIL resistance, i.e., presence of cIAP-1 protein in SK-OV-3^{S} cells leads to TRAIL resistance which can be overcome by the addition of a cIAP-1 Antagonist compound that binds cIAP-1 in combination with TRAIL while degradation of cIAP-1 in SK-OV-3^{R} cells renders them sensitive to TRAIL. In this way, an cIAP-1 Antagonist that binds cIAP-1 acts synergistically with TRAIL.

For simplicity and illustrative purposes, the principles of the invention are described by referring to illustrative embodiments thereof. In addition, in the preceding and following description, numerous specific details are set forth in order to provide a thorough understanding of the invention. It will be apparent however, to one of ordinary skill in the art, that the invention may be practiced without limitation to these specific details. In other instances, well known methods and structures have not been described in detail so as not to unnecessarily obscure the invention.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

The present invention is directed generally to the use of Smac mimetics that have affinity for cIAP-1, which affinity is preferably greater than for XIAP.

In an embodiment of the invention, cIAP-lbinding affinity data are submitted to a regulatory agency as part of a dossier for seeking approval to conduct human clinical trials with a cIAP-1 Antagonist. In the United States, such approval is referred to as an IND or an IND exemption, because it is an exemption, for an investigational new drug, from laws that prohibit administration of unapproved drugs to humans. Such binding data can also include absolute or relative binding affinities for other IAPB, e.g., XIAP. In certain embodiments, such data show that binding of a given agent for which the approval is being sought is greater for cIAP-1 than for XIAP, as discussed elsewhere in this specification.

Alternatively, or in addition to such data, an entity seeking such approval (or exemption) can provide data showing degradation of cIAP-1. Such data could also include data showing relative or absolute degradation of other IAPB, such as XIAP.

Alternatively, or in addition, such binding data, degradation data, or both can be submitted to a regulatory agency to support an application for approval to market a cIAP-1 Antagonist. For example, such data can be submitted as a part of a New Drug Approval Application (NDA) with the United States Food and Drug Administration (FDA).

Alternatively, or in addition, such binding data, degradation data, or both can be used as go-no go decision points in drug discovery and development. For example, a compound can be selected for further development based on whether or not it exhibits binding to cIAP-1 and/or degradation of a cIAP-1. As discussed elsewhere in this specification, such binding affinity can be greater than for other IAPB and the rate of degradation can be faster than for that of other IAPB.

Alternatively, or in addition, such data can be used to characterize a given agent that has been selected for further development based on other data, such as cell toxicity data.

In any event, binding to cIAP-1 or other IAPB can be determined using standard binding affinity assays, as illustrated above. Crystallization of a full-length Smac protein with XIAP˜BIR3 and NMR spectroscopy of an N-terminal Smac 9-mer peptide with the BIR3 domain of XIAP has revealed that Smac N-terminal AVPI residues are critical for binding to XIAP. Homologous residues in processed caspase 9 and other proteins define these four residues as the “IAP binding motif”. Peptides bearing this configuration have been shown to bind to XIAP at the same site as the N-terminal ATPF of the p12 subunit of active caspase 9, thereby relieving XIAP inhibition of caspase 9 and allowing apoptosis to proceed. We have utilized the specificity of this IAP binding motif in a fluorescence polarization assay to measure the binding affinities for cIAP-1 Antagonists. The fluorescence polarization assay consists of FP peptide {Sri: What is “FP Peptide”?}, and the recombinant BIR3 domain of the XIAP protein. The FP peptide and mimics of cIAP-1 N-terminus compete for binding to the BIR3 protein. However, if the compound does not compete with the FP peptide, the labeled peptide remains bound to the BIR3 and there is a high mP (millipolarization) value. If a peptide, peptidomimetic, or other small molecule being tested is a competitor, then it succeeds in displacing the FP peptide, resulting in a low mP value. Molecules that compete with the FP peptide can be titrated and IC₅₀ values determined (GraphPad Prism nonlinear regression curve-fitting program) by plotting mp value as the direct measure of fraction bound vs. the log of the compound concentration.

Similarly, IAP degradation assays can be carried out by well known techniques, as illustrated above. Comparable to protein phosphorylation, ubiquitination is a reversible processes, regulated by the activities of E3 protein ubiquitin ligases which function to covalently attach ubiquitin molecules to target proteins. cIAP-1 contains a c-terminal ring domain that enables cIAP-1 to catalyze itself and selected target proteins. Ubiquitinated protein is then escorted to the 26S proteasome where it undergoes final degradation and the ubiquitin is released and recycled. Once cIAP-1 Antagonists bind to cIAP-1, it results in perturbation of cell survival complexes or dissociation of natural ligands, signaling IAPB to either self ubiquinate or become targets for ubiquitination followed by proteasomal degradation. As previously mentioned, western blot analysis of cell lysate after cIAP-1 Antagonist treatment resulted in disappearance of cIAP-1 and XIAP bands when compared to no drug treatment. To further elucidate the machinery involved with this phenomenon, we focused on the regulation of IAP stability and asked whether or not the proteasome was involved in the degradation of cIAP-1 and XIAP. We found that addition of botezomib to cells during cIAP-1 Antagonist treatment completely prevented cIAP-1 and XIAP degradation as detected by western blotting. This experiment suggests that cIAP-1 and XIAP are ubiquitinated and targeted for proteasome degradation.

Preferably, following internal administration to a human (or other animal) suffering a proliferative disorder, such cIAP-1 Antagonist causes degradation of cIAP-1. Preferably, the cIAP-1 Antagonist is selected to be one which causes such degradation to occur more quickly than degradation of XIAP, as discussed above.

In one embodiment the cIAP-1 Antagonists act as chemopotentiating agents. The term “chemopotentiating agent” refers to an agent that acts to increase the sensitivity of an organism, tissue, or cell to a chemical compound, or treatment namely “chemotherapeutic agents” or “chemo drugs” or radiation treatment. A further embodiment of the invention is a pharmaceutical composition of a cIAP-1 Antagonist, which can act as a chemopotentiating agent, and a chemotherapeutic agent or chemoradiation. Another embodiment of the invention is a method of inhibiting tumor growth in vivo by administering such cIAP-1 Antagonist. Another embodiment of the invention is a method of inhibiting tumor growth in vivo by administering a chemopotentiating cIAP-1 Antagonist and a chemotherapeutic agent or chemoradiation. Another embodiment of the invention is a method of treating a patient with a cancer by administering cIAP-1 Antagonists of the present invention alone or in combination with a chemotherapeutic agent or chemoradiation.

In an embodiment of the invention a therapeutic composition, i.e., a pharmaceutical composition, for promoting apoptosis can be a therapeutically effective amount of a cIAP-1 Antagonist which binds to at least one IAP other than a cIAP. In another embodiment the IAP can be XIAP. Any of the aforementioned therapeutic compositions may further include a pharmaceutical carrier.

Embodiments of the invention also include a method of treating a patient with a condition in need thereof wherein a therapeutically effective amount of a cIAP-1 Antagonist is delivered to the patient, and the cIAP-1 Antagonist binds to cIAP-1. Embodiments of the invention also include a method of treating a patient with cancer by promoting apoptosis by administration of an effective amount of a cIAP-1 Antagonist, and the cIAP-1 Antagonist binds to cIAP-1.

Embodiments of the invention also include a method of treating a patient with an autoimmune disease by administration of an effective amount of a cIAP-1 Antagonist.

In each of the above illustrative embodiments, the composition or method may further include a chemotherapeutic agent. The chemotherapeutic agent can be, but is not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-estrogenic analogs.

The invention disclosed herein provides methods and compositions for enhancing apoptosis in pathogenic cells. The general method comprises contacting the cells with an effective amount of a cIAP-1 Antagonist.

In some embodiments, the cells are in situ in an individual and the contacting step is affected by administering to the individual a pharmaceutical composition comprising an effective amount of the cIAP-1 Antagonist wherein the individual may be subject to concurrent or antecedent radiation or chemotherapy for treatment of a neoproliferative pathology. The pathogenic cells are of a tumor such as, but not limited to, breast cancer, prostate cancer, lung cancer, pancreatic cancer, gastric cancer, colon cancer, ovarian cancer, renal cancer, hepatoma, melanoma, lymphoma, and sarcoma.

In addition to apoptosis defects found in tumors, defects in the ability to eliminate self-reactive cells of the immune system due to apoptosis resistance are considered to play a key role in the pathogenesis of autoimmune diseases. Autoimmune diseases are characterized in that the cells of the immune system produce antibodies against its own organs and molecules or directly attack tissues resulting in the destruction of the latter. A failure of those self-reactive cells to undergo apoptosis leads to the manifestation of the disease. Defects in apoptosis regulation have been identified in autoimmune diseases such as systemic lupus erythematosus or rheumatoid arthritis.

The subject compositions encompass pharmaceutical compositions comprising a therapeutically effective amount of a cIAP-1 Antagonist in a dosage form with a pharmaceutically acceptable carrier, wherein the cIAP-1 Antagonist inhibits the activity of an Inhibitor of Apoptosis protein, thus promoting apoptosis. Another embodiment of the present invention are compositions comprising a therapeutically effective amount of a cIAP-1 Antagonist in dosage form and a pharmaceutically acceptable carrier, in combination with a chemotherapeutic and/or radiotherapy, wherein the cIAP-1 Antagonist inhibits the activity of an Inhibitor of Apoptosis protein (IAP), thus promoting apoptosis and enhancing the effectiveness of the chemotherapeutic and/or radiotherapy.

Administration of clAP-1 Antagonists. The cIAP-1 Antagonists are administered in effective amounts. An effective amount is that amount of a preparation that alone, or together with further doses, produces the desired response. This may involve only slowing the progression of the disease temporarily, although preferably, it involves halting the progression of the disease permanently or delaying the onset of or preventing the disease or condition from occurring. This can be monitored by routine methods. Generally, doses of active compounds would be from about 0.01 mg/kg per day to 1000 mg/kg per day. It is expected that doses ranging from 50-500 mg/kg will be suitable, preferably intravenously, intramuscularly, or intradermally, and in one or several administrations per day. The administration of the cIAP-1 Antagonist can occur simultaneous with, subsequent to, or prior to chemotherapy or radiation so long as the chemotherapeutic agent or radiation sensitizes the system to the cIAP-1 Antagonist.

In general, routine experimentation in clinical trials will determine specific ranges for optimal therapeutic effect for each therapeutic agent and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the cIAP-1 Antagonist potencies, the duration of the treatment and the severity of the disease being treated. For example, a dosage regimen of the cIAP-1 Antagonist can be oral administration of from 1 mg to 2000 mg/day, preferably 1 to 1000 mg/day, more preferably 50 to 600 mg/day, in two to four (preferably two) divided doses, to reduce tumor growth. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient tolerance permits. Multiple doses per day are contemplated to achieve appropriate systemic levels of compounds. Generally, a maximum dose is used, that is, the highest safe dose according to sound medical judgment. Those of ordinary skill in the art will understand, however, that a patient may insist upon a lower dose or tolerable dose for medical reasons, psychological reasons or for virtually any other reason.

Routes of administration. A variety of administration routes are available. The particular mode selected will depend, of course, upon the particular chemotherapeutic drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes are particularly suitable for purposes of the present invention.

In one aspect of the invention, a cIAP-1 Antagonist as described herein, with or without additional chemotherapeutic agents or radiotherapy, does not adversely affect normal tissues, while sensitizing tumor cells to the additional chemotherapeutic/radiation protocols. While not wishing to be bound by theory, it would appear that because of this tumor specific induced apoptosis, marked and adverse side effects such as inappropriate vasodilation or shock are minimized. Preferably, the composition or method is designed to allow sensitization of the cell or tumor to the chemotherapeutic or radiation therapy by administering at least a portion of the cIAP-1 Antagonist prior to chemotherapeutic or radiation therapy. The radiation therapy, and/or inclusion of chemotherapeutic agents, may be included as part of the therapeutic regimen to further potentiate the tumor cell killing by the cIAP-1 Antagonist.

Pharmaceutical compositions. In one embodiment of the invention, an additional chemotherapeutic agent (infra) or radiation may be added prior to, along with, or following the cIAP-1 Antagonist. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The delivery systems of the invention are designed to include time-released, delayed release or sustained release delivery systems such that the delivering of the cIAP-1 Antagonist occurs prior to, and with sufficient time, to cause sensitization of the site to be treated. A cIAP-1 Antagonist may be used in conjunction with radiation and/or additional anti-cancer chemical agents. Such systems can avoid repeated administrations of the cIAP-1 Antagonist, increasing convenience to the subject and the physician, and may be particularly suitable for certain compositions of the present invention.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous preparation of a chemopotentiating agent (e.g. cIAP-1 Antagonist), which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA which is incorporated herein in its entirety by reference thereto.

Additional chemotherapeutic agents. Chemotherapeutic agents suitable, include but are not limited to the chemotherapeutic agents described in “Modern Pharmacology with Clinical Applications”, Sixth Edition, Craig & Stitzel, Chpt. 56, pg 639-656 (2004), herein incorporated by reference. This reference describes chemotherapeutic drugs to include alkylating agents, antimetabolites, anti-tumor antibiotics, plant-derived products such as taxanes, enzymes, hormonal agents such as glucocorticoids, miscellaneous agents such as cisplatin, monoclonal antibodies, immunomodulating agents such as interferons, and cellular growth factors. Other suitable classifications for chemotherapeutic agents include mitotic inhibitors and nonsteroidal anti-estrogenic analogs. Other suitable chemotherapeutic agents include toposiomerase I and II inhibitors: CPT (8-Cyclopentyl-1,3-dimethylxanthine, topoisomerase I inhibitor) and VP16 (etoposide, topoisomerase II inhibitor).

Specific examples of suitable chemotherapeutic agents include, but are not limited to, cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, TRAIL, interferon (in both its alpha and beta forms), thalidomide, and melphalan. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim and sargramostim. Chemotherapeutic compositions also comprise other members, i.e., other than TRAIL, of the TNF superfamily of compounds.

Radiotherapy protocols. Additionally, in several method embodiments of the present invention the cIAP-1 Antagonist therapy may be used in connection with chemo-radiation or other cancer treatment protocols used to inhibit tumor cell growth.

For example, but not limited to, radiation therapy (or radiotherapy) is the medical use of ionizing radiation as part of cancer treatment to control malignant cells is suitable for use in embodiments of the present invention. Although radiotherapy is often used as part of curative therapy, it is occasionally used as a palliative treatment, where cure is not possible and the aim is for symptomatic relief. Radiotherapy is commonly used for the treatment of tumors. It may be used as the primary therapy. It is also common to combine radiotherapy with surgery and/or chemotherapy. The most common tumors treated with radiotherapy are breast cancer, prostate cancer, rectal cancer, head & neck cancers, gynecological tumors, bladder cancer and lymphoma. Radiation therapy is commonly applied just to the localized area involved with the tumor. Often the radiation fields also include the draining lymph nodes. It is possible but uncommon to give radiotherapy to the whole body, or entire skin surface. Radiation therapy is usually given daily for up to 35-38 fractions (a daily dose is a fraction). These small frequent doses allow healthy cells time to grow back, repairing damage inflicted by the radiation. Three main divisions of radiotherapy are external beam radiotherapy or teletherapy, brachytherapy or sealed source radiotherapy, and unsealed source radiotherapy, which are all suitable examples of treatment protocol in the present invention. Administration of the cIAP-1 Antagonist may occur prior to, after, or concurrently with the treatment protocol.

The above describes illustrative embodiments of the invention. However, the invention is not limited to the precise aspects described above but rather includes modifications thereof and alternatives thereto that come within the scope of the following claims.

1-12. (canceled)

13. A method of treating a proliferative disorder in a subject that comprises administering to the subject an effective amount of a compound that binds to cIAP-1 .

14. The method of claim 13 wherein the compound binds preferentially to cIAP-1, relative to XIAP.

15. The method of claim 13 wherein the compound is a Smac peptidomimetic.

16. A pharmaceutical composition comprising a cIAP-1 Antagonist that preferentially binds cIAP-1 relative to XIAP, and a pharmaceutically acceptable carrier.

17. The pharmaceutical composition of claim 16 comprising an effective amount of the cIAP-1 Antagonist that is less than the effective amount of an XIAP antagonist.

18-19. (canceled)

20. A method of treating a proliferative disorder in a human or animal subject, which proliferative disorder is mediated primarily by cIAP-1 activity, which comprises administering to the subject an effective amount of a compound that binds preferentially to cIAP-1 relative to XIAP.

21-22. (canceled)
